(19) United States
(12) Patent Application Publication
(10) Pub. No.: US 2025/0093348 A1
(43) Pub. Date: Mar. 20, 2025

(54) NUCLEIC ACID TESTING DEVICE AND METHOD

(71) Applicant: ZHEJIANG UNIVERSITY, Zhejiang (CN)

(72) Inventors: Jian WU, Zhejiang (CN); Yanju CHEN, Zhejiang (CN)

(73) Assignee: ZHEJIANG UNIVERSITY, Zhejiang (CN)

(21) Appl. No.: 18/565,522

(22) PCT Filed: Nov. 4, 2022
(86) PCT No.: PCT/CN2022/130016

§ 371 Date: Nov. 30, 2023

(30) Foreign Application Priority Data
Jun. 23, 2022 (CN) 202210724667.0

(51) Int. Cl. G01N 33/543; B01L 7/00

(52) U.S. Cl. CPC G01N 33/54388; B01L 7/52; B01L 2200/0689; B01L 2200/141; B01L 2200/16; B01L 2300/0825; B01L 2300/123; B01L 2300/18; B01L 2400/0688

(57) ABSTRACT

A nucleic acid testing device and method are provided. A base plate that is turned during nucleic acid testing is included, wherein the base plate includes the following chambers: a sample chamber for adding and containing a nucleic acid sample solution, the nucleic acid sample solution is placed in the chamber, and an upper port of the sample chamber communicates with the outside atmosphere; a reaction chamber for nucleic acid amplification reaction and located below the sample chamber, a nucleic acid amplification reagent for nucleic acid amplification reaction is pre-placed in the chamber, and an upper end of the reaction chamber communicates with a bottom end of the sample chamber though a longitudinal liquid channel; a test strip chamber for lateral flow test strip testing, a nucleic acid test strip is pre-placed in the chamber, and the test strip chamber communicates with the reaction chamber through a bent channel.

### BACKGROUND OF THE INVENTION

### Technical Field

The disclosure belongs to a nucleic acid testing method and device in the field of biochemical analysis, and in particular, relates to a nucleic acid testing method and device capable of testing a nucleic acid amplification product by using a lateral flow test strip. Description of Related Art

Regarding the test strips for lateral flow nucleic acid testing, a number of reports, such as the related document of “Rapid developments in lateral flow immunoassay for nucleic acid testing” (Analyst, 2021, 146, 1514-1528), can be found.

Using lateral flow nucleic acid test strips to test target nucleic acid is a simple visual testing method, and such a method may facilitate the promotion of nucleic acid testing to grassroots and families, and thus, may be applied in various fields such as disease diagnosis, food safety, and environmental testing. The lateral flow nucleic acid test strips can be used in combination with polymerase chain reaction (PCR) and various isothermal amplification methods, such as LAMP, RPA, etc.

However, the combination of nucleic acid amplification method and the lateral flow test strip still exhibits several shortcomings at present and needs to be further improved. The main problem is that the nucleic acid amplification products need to be added to the sample pad of the test strip during the operation. In order to complete such an operation, if the amplification process is performed in a separate reaction tube, the amplification tube needs to be opened after the nucleic acid amplification is completed. Such an opening process can easily cause aerosol contamination of the amplification product in the environment, which may lead to a false positive result in subsequent tests. Further, due to the high sensitivity of nucleic acid amplification testing, in order to reduce the consumption of reagents, the volume of the reaction system for nucleic acid amplification is small generally. However, the amount of solution required by the common lateral flow test strip assay is greater than that required by the ordinary nucleic acid amplification reaction system. As such, a dilution step of the amplification product is involved in the entire testing process. This dilution step also increases the complexity of the overall operation.

At present, in some methods, the amplification and test strips are integrated into one device. Such integration, on the one hand, prevents the release of aerosols into the surrounding environment, and on the other hand, it also enables the entire process of nucleic acid amplification, product dilution, and solution addition to the sample pad of the test strip. However, these methods and devices are relatively complex and costly. In view of these problems, the disclosure provides a new nucleic acid testing method and a corresponding device capable of implementing nucleic acid amplification and test strip testing.

### SUMMARY OF THE INVENTION

To solve the problems mentioned in the BACKGROUND, the disclosure provides a nucleic acid testing method and device capable of achieving nucleic acid amplification and lateral flow testing of its product quickly and effectively at low costs and without contamination, so that the problem of lack of training and experience in grassroots and home testing is avoided. The disclosure includes the following technical solutions.

### I. A Nucleic Acid Testing Device

A base plate that is turned during nucleic acid testing is included, and the base plate is turned in a clockwise direction towards a bent channel. Specifically, when a channel connecting a sample chamber and a test strip chamber is on a left side of the sample chamber, the base plate is turned counterclockwise. When the channel connecting the sample chamber and the test strip chamber is on a right side of the sample chamber, the base plate is turned clockwise. Similarly, when a channel connecting a reaction chamber and the test strip chamber is on a left side of the reaction chamber, the base plate is turned counterclockwise. When the channel connecting the reaction chamber and the test strip chamber is on a right side of the reaction chamber, the base plate is turned clockwise. The base plate includes the following chambers as described in the following paragraphs.

A sample reaction chamber for adding and containing a nucleic acid sample solution and a nucleic acid amplification reagent is included. The nucleic acid sample solution and the nucleic acid amplification reagent are placed in the chamber.

A test strip chamber for lateral flow nucleic acid test strip testing is included. A nucleic acid test strip is pre-placed in the chamber, the test strip chamber communicates with a sample chamber through a bent channel, and at least one point in the bent channel is higher than a highest liquid level for a solution to be added in the sample reaction chamber/sample chamber. Further, all channels are located on the left or right side of the sample chamber. The nucleic acid test strip is attached to a corner or an inner wall of the test strip chamber close to the bent channel.

The sample reaction chamber is specifically divided into the sample chamber and the reaction chamber.

The sample chamber for adding and containing the nucleic acid sample solution is included, and the nucleic acid sample solution is placed in the chamber.

The reaction chamber for nucleic acid amplification reaction and located below the sample chamber is included. The nucleic acid amplification reagent for nucleic acid amplification reaction is pre-placed in the chamber. The test strip chamber communicates with the sample chamber through the reaction chamber. The test strip chamber does not directly communicate with the sample chamber. An upper end of the reaction chamber communicates with a bottom end of the sample chamber though a liquid channel. The test strip chamber communicates with the reaction chamber through the bent channel.

A volume of the reaction chamber is less than 50 microliters. When a liquid volume of the amplification reaction is greater than 50 microliters, the nucleic acid amplification reagent used for the nucleic acid amplification reaction can be placed in the sample chamber. The sample chamber is directly used for the amplification reaction and the reaction chamber is omitted. In this case, the sample chamber can also be called a sample reaction chamber.

The bent channel is a U-shaped, Z-shaped, S-shaped, inverted U-shaped, or L-shaped channel.

The bent channel is one or a combination of a straight channel and a curved channel.

A capillary valve for controlling flowing and a stop position of a liquid is provided in the bent channel. A position of the capillary valve is set as: when the device is in a horizontal state, a solution static pressure in the bent channel cannot break through a resistance of the capillary valve, and when the device is in a non-horizontal state, position changes of the sample reaction chamber/sample chamber and the reaction chamber cause the solution static pressure in the bent channel to increase, so that the solution static pressure in the bent channel breaks through the capillary valve and causes the liquid to flow in the bent channel.

A gas communication channel is also included, and the test strip chamber communicates with a top portion of the sample reaction chamber/sample chamber through the gas communication channel.

A reagent chamber for accommodating a nucleic acid testing reagent and at a same height as the sample reaction chamber/sample chamber is also included. A testing reagent is added to the chamber, and the testing reagent is a labeled single-stranded DNA sequence. A lower end of the reagent chamber communicates with the bent channel between the test strip chamber and the sample reaction chamber/reaction chamber after passing through the liquid channel. At least one point in the liquid channel is higher than the highest liquid level for a solution to be added in the sample reaction chamber/sample chamber and a highest liquid level for a solution to be added in the reagent chamber, and the high point of the two is used as a comparison point.

A gas communication channel is also included, and the test strip chamber communicates with top portions of the sample reaction chamber/sample chamber and the reagent chamber after passing through the gas communication channel. Specifically, a vertical gas communication channel and a horizontal gas communication channel are arranged. The horizontal gas communication channel communicates the sample chamber and the top portion of the reagent chamber, and the vertical gas communication channel communicates the top portion of the reagent chamber and the test strip chamber.

Each of the sample reaction chamber/sample chamber and the reagent chamber is provided with a partition structure for preventing the solution in the chamber from entering a communication channel at a top portion of the chamber when the chamber is turned.

The first longitudinal liquid channel, the second longitudinal liquid channel, and the third longitudinal liquid channel are all located on the same side of the reaction chamber. The channels are also located on the same side of the reagent chamber, the sample chamber, and the reaction chamber, and the reagent chamber and the sample chamber are located on the other side of the reaction chamber.

A pneumatic buffer chamber for anti-contamination is also included. The pneumatic buffer chamber communicates with the gas communication channel, so that the pneumatic buffer chamber directly communicates with the sample chamber, the reagent chamber, and the test strip chamber. An opening is provided on one side wall of the pneumatic buffer chamber, and a flexible film is arranged to seal the opening.

The reaction chamber is provided with a temperature control device for heating the reaction chamber. When the reaction chamber is omitted, the temperature control device can be located at the bottom portion of the sample chamber.

### II. The First Nucleic Acid Testing Method of the Abovementioned Nucleic Acid Testing Device

In S1, the nucleic acid amplification reagent and the nucleic acid sample solution are pre-added to the sample reaction chamber, and the nucleic acid amplification reaction is directly performed in the sample reaction chamber.

In S3, a reaction amplification liquid is obtained in the nucleic acid sample solution after the amplification reaction is completed, and the entire device is turned by an angle in a clockwise direction where a bent channel is arranged. The reaction amplification liquid and the solution in the reagent chamber enter a bottom portion in the test strip chamber after passing through the bent channel under the action of a static pressure and are mixed, so that a labeled single-stranded DNA sequence in the testing reagent is combined with a target nucleic acid sequence in the reaction amplification liquid, and after reaching the bottom portion of the test strip chamber, the mixed solution contacts the nucleic acid test strip in the test strip chamber for immunoassay reaction and testing.

### III. The Second Nucleic Acid Testing Method of the Abovementioned Nucleic Acid Testing Device

In S1, the nucleic acid amplification reagent is pre-added to the reaction chamber, the nucleic acid sample solution is pre-added to the sample chamber, and a testing reagent is pre-added to the reagent chamber.

In S2, the nucleic acid sample solution is added to the sample chamber. The nucleic acid sample solution enters the reaction chamber under the action of a static pressure, and the nucleic acid amplification reaction is performed in the reaction chamber. The action of the static pressure is ensured by the capillary channel.

In S3, a reaction amplification liquid is obtained in the reaction chamber after the amplification reaction is completed, and the base plate of the entire device is turned by an angle in a clockwise direction where a bent channel is arranged. The solutions in the reaction chamber, the sample chamber, and the reagent chamber enter a bottom portion in the test strip chamber after passing through a bent channel under the action of the static pressure and are mixed, so that a labeled single-stranded DNA sequence in the testing reagent is combined with a target nucleic acid sequence in the reaction amplification liquid, and after reaching the bottom portion of the test strip chamber, the mixed solution contacts the nucleic acid test strip in the test strip chamber for immunoassay reaction and testing. Specifically, when the channel connecting the reaction chamber and the test strip chamber is on the left side of the reaction chamber, it is turned counterclockwise. When the channel connecting the reaction chamber and the test strip chamber is on the right side of the reaction chamber, it is turned clockwise.

Beneficial effects provided by the disclosure include the following.

Nucleic acid amplification and lateral flow testing of its product can be implemented through the device and method provided by the disclosure. To complete the testing, the user has only two steps to perform. The first is to drop a specific amount of sample to be measured into the sample chamber 10. The second is to turn the device after the nucleic acid amplification is completed. This turning operation can also be achieved by using supporting instruments, and the turning can be achieved conveniently and cost-effectively by using a steering engine. These lay the foundation for the promotion of nucleic acid testing to grassroots and family use.

Further, in the device of the disclosure, after the sample is added, it is only necessary to tightly cover the mouth of the sample chamber. The inside of this device is completely isolated from the outside, without any gas or liquid connections. Further, the device is also provided with the pneumatic buffer chamber. The internal air pressure of the device placed under heating conditions is higher than the external air pressure, which has a strong anti-contamination capability. The entire device achieves the automatic balance of the air pressure in each chamber through the design of the internal air passage. In this way, liquid movement can be controlled by hydrostatic pressure without any external pumps or valves, and there is no connection with the outside. As far as nucleic acid amplification is concerned, the anti-contamination design of nucleic acid amplicons is important. Especially for grassroots and household testing, operators often lack training and experience, and the anti-contamination design is thus more important. The device provided by the disclosure therefore has advantages herein.

Compared to the currently-available methods, the device and method provided by the disclosure exhibit the advantages of a simple structure, convenient operation, stable results, and prevention of contamination with nucleic acid amplification products.

### BRIEF DESCRIPTION OF THE DRAWINGS

FIG. 1 is a schematic structural view of a device according to the disclosure. 1: base plate: 21, 22, 23, 24: longitudinal liquid channel: 31, 32, 33: transverse liquid channel; 4: reaction chamber; 5: test strip chamber; 61, 62: gas communication channel; 7: pneumatic buffer chamber: 8: reagent chamber; 91, 92: in-chamber dividing plate; 10: sample chamber; 11: capillary channel; and 121, 122: capillary valve.

FIG. 2 is a schematic view of the device according to the disclosure, a reaction chamber and a pneumatic buffer chamber can be omitted, where 10 is labeled as a sample reaction chamber, and the remaining labels can be seen in FIG. 1.

FIG. 3 is a schematic cross-sectional view of a pneumatic buffer chamber 7. 13: flexible film.

FIG. 4 is a result of a nucleic acid lateral flow test strip testing. a) positive sample, b) negative sample.

FIG. 5 is a schematic view of the device according to an embodiment of the disclosure, and no reagent chamber is arranged in the device.

FIG. 6 is a schematic view of the device according to an embodiment of the disclosure. 14: steering engine interface, 15: temperature control device.

### DETAILED DESCRIPTION OF THE EMBODIMENTS

The disclosure is further described together with accompanying drawings and specific embodiments in the following paragraphs.

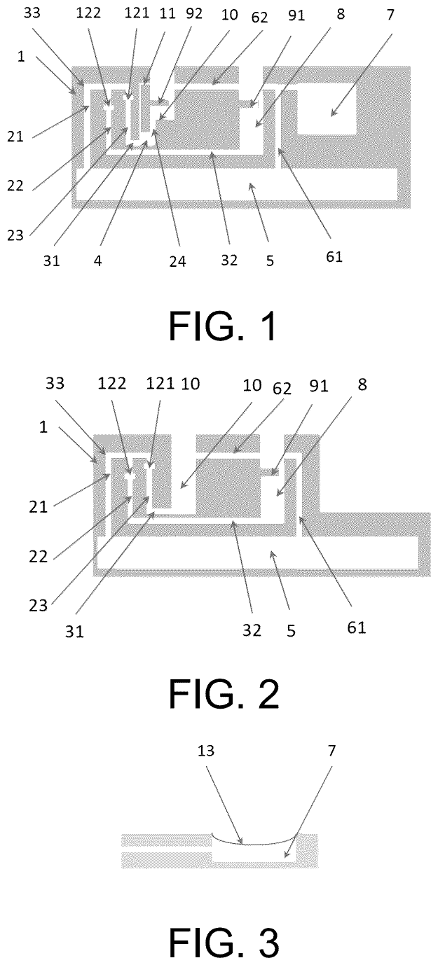

The method and supporting equipment of the disclosure are introduced through FIG. 1. Please note that structural details are highlighted for convenience of illustrating relevant aspects of the disclosure, and the drawing is not drawn to scale based on the actual dimensions of each structure.

During description, the up and down direction of the paper surface is the vertical direction (i.e., up and down or high and low are used in the text to describe the relative positions), the horizontal direction of the paper surface is the transverse direction (i.e., left and right are used in the text to describe the relative positions), and the direction perpendicular to the paper surface is the depth direction (i.e., concave and convex or deep and shallow are used in the text to describe the relative positions).

The left and right positions described in the following paragraphs are all taken as an example for the device to be turned counterclockwise for testing under specific conditions. The device may also be turned clockwise, and in this case, all described left and right positions shall be interchanged.

As shown in FIG. 1, a device includes five independent chambers arranged inside a base plate 1, namely, a sample chamber 10, a reagent chamber 8, a reaction chamber 4, a test strip chamber 5, and a pneumatic buffer chamber 7. A volume of the sample chamber 10 is generally greater than 50 microliters, and a volume of the reaction chamber 4 is less than 50 microliters. When amplification reaction is performed on a large sample volume (greater than 50 microliters), that is, when a liquid volume of the amplification reaction is greater than 50 microliters, the sample chamber 10 and the reaction chamber 4 may be integrated into one chamber, which is called a sample reaction chamber (as shown in FIG. 2).

A side surface of the base plate 1 is connected to an output shaft of a steering engine. Specifically, a steering engine interface 14 for connecting the output shaft of the steering engine is provided on the side surface of the base plate 1, and the output shaft of the steering engine is inserted into the steering engine interface 14 to drive the base plate 1 to be turned.

The reaction chamber 4 is located at the lower left of the sample chamber 10, and the two are connected through a channel 24. The reaction chamber 4 is connected to the test strip chamber 5 through a liquid channel of a bent channel. From the perspective of the vertical direction, at least one point in the bent channel is higher than a highest liquid level for a solution to be added in the sample chamber 10. Further, from the perspective of the horizontal direction, the bent channel is located on one side of the reaction chamber, and from the perspective of the horizontal direction, the reaction chamber 4 is located between the sample chamber 10 and the bent channel. When the reaction chamber 4 is omitted, the sample reaction chamber formed by integrating the sample chamber 10 and the reaction chamber 4 is connected to the test strip chamber 5 through the liquid channel in the same manner.

The bent channel is a U-shaped, Z-shaped, S-shaped, inverted U-shaped, or L-shaped channel. Further, the bent channel is one or a combination of a straight channel and a curved channel.

For example, a specific form of the bent channel connecting the reaction chamber 4 and the test strip chamber 5 is formed by a nearly U-shaped straight channel. The reaction chamber 4 communicates with the test strip chamber 5 through an inverted U-shaped bent channel formed by a horizontal first transverse liquid channel 31, a vertical third longitudinal liquid channel 23, a horizontal third transverse liquid channel 33, a vertical first longitudinal liquid channel 21 in sequence. The third transverse liquid channel 33 is higher than the reaction chamber 4, where a top end of the third longitudinal liquid channel 23 is higher than the highest liquid level for a solution to be added in the sample chamber 10.

That is, as shown in FIG. 1, left and lower portions of the reaction chamber 4 pass through the channel 31 and further pass through the channel 23, the channel 33, and the channel 21 to communicate with the test strip chamber 5. The channel 23 is arranged such that a highest longitudinal end of the channel 23 is higher than the highest liquid level for a solution to be added in the sample chamber 10. That is, when the device is in the horizontal position as shown in FIG. 1, the solution added to the sample chamber 10 may not fill the channel 23, so as to prevent a solution from entering the test strip chamber 5 under the condition of this position. When the reaction chamber 4 is omitted, the sample chamber 10 is connected to the test strip chamber 5 through the liquid channel in the same manner, as shown in FIG. 2.

A pre-solidified nucleic acid amplification reagent is provided in the reaction chamber 4, which can be solidified by freeze-drying, air-drying and other methods. As long as the corresponding sample solution is added, together with appropriate temperature control, the nucleic acid amplification reaction can be completed. When a volume of the nucleic acid amplification reaction is greater than 50 microliters, the reaction chamber 4 may be omitted, and the nucleic acid amplification reagent may be pre-solidified at the bottom of the sample reaction chamber.

The reagent chamber 8 is located on a left side or a right side of the sample chamber 10 and is basically at a same height as the sample chamber 10. A lower end of the reagent chamber 8 communicates with the bent channel between the test strip chamber 5 and the reaction chamber 4 after passing through the liquid channel. At least one point in the liquid channel is higher than the highest liquid level for a solution to be added in the sample chamber 10 and a highest liquid level for a solution to be added in the reagent chamber 8, and the high point of the two is used as a comparison point.

For instance, the lower end of the reagent chamber 8 communicates with the middle of the third transverse liquid channel 33 after passing through a horizontal second transverse liquid channel 32 and a vertical second longitudinal liquid channel 22 in sequence. Both ends of the third transverse liquid channel 33 communicate with the first longitudinal liquid channel 21 and the third longitudinal liquid channel 23. A top end of the second longitudinal liquid channel 22 is higher than the highest liquid level for a solution to be added in the reagent chamber 8 and the highest liquid level for a solution to be added in the sample chamber 10.

As specifically shown in FIG. 1, the left and lower portions pass through the channel 32 and further pass through the channel 22, the channel 33, and the channel 21 to communicate with the test strip chamber 5. The channel 22 is arranged such that a highest longitudinal end of the channel 22 is higher than a solution level in the reagent chamber 8. That is, when the device is in the horizontal position as shown in FIG. 1, the solution added to the reagent chamber 8 may not fill the channel 22, so as to prevent a solution from entering the test strip chamber 5 under the condition of this position. Further, this design can also ensure that the solution in the reagent chamber 5 may not mix with the solutions in the sample chamber 10 and the reaction chamber 4 when the device is placed horizontally as shown in the figure.

A capillary channel 11 may also be provided between the reaction chamber 4 and the sample chamber 10. A top portion of the test strip chamber 5 communicates with the reaction chamber 4 through the capillary channel 11. A top end of the capillary channel 11 is higher than the highest liquid level for a solution to be added in the sample chamber 10. The function of the capillary channel 11 is to prevent bubbles from forming on the top portion of the reaction chamber 4 when a liquid enters the reaction chamber 4 from the sample chamber 10 under the action of static pressure.

The sample chamber 10 and the reagent chamber 8 are provided with partition structures 91 and 92 as shown in the figure. Therefore, when the device is turned, the solution in the reagent chamber 8 may be prevented from entering the channel 62, and the solution in the sample chamber 10 may be prevented from entering the capillary channel 11. The partition structure 91 is disposed on an inner wall of the top portion of the reagent chamber 8 away from the channel 62 and is located below the channel 62. The partition structure 92 is disposed on an inner wall of the top potion of the sample chamber 10 on the side away from the capillary channel 11 and is located below the capillary channel 11.

Positions of the channels 21, 22, and 23 are all on the left side of the reagent chamber 8. the sample chamber 10, and the reaction chamber 4, and the channel 21 is leftmost with respect to the channels 22 and 23. The channel 33 connects highest longitudinal ends (top portions) of the channel 21, the channel 22, and the channel 23.

A capillary valve for controlling flowing and a stop position of a liquid is arranged in the liquid channel connecting the reaction chamber 4 and the test strip chamber 5. The arrangement principle of the capillary valve position is: when the device is in a horizontal state, a solution static pressure in the channel cannot break through a resistance of the capillary valve. When the device is turned, the solution static pressure in the channel increases due to the position changes of the sample chamber 10 and the reaction chamber 4, so that the capillary valve can be broken through, causing the liquid to flow in the channel.

FIG. 1 shows a specific form of arrangement of the capillary valves in the channels 22 and 23. A capillary valve 121 is arranged at a position higher than the highest liquid level for a solution to be added in the sample chamber 10. A capillary valve 122 is arranged at a position higher than the highest liquid level for a solution to be added in the reagent chamber 8. When the device is placed horizontally as shown in the figure, the liquid in the reaction chamber 4 reaches the position of the capillary valve under the action of capillary force. Due to the change in the direction of surface tension at the position of the capillary valve, the liquid flow stops at the position of the capillary valve. After the nucleic acid amplification reaction is completed, since the device is turned by a specific angle, the liquid in the channel is not only affected by surface tension, but also by the hydrostatic pressure in the channel. Under the action of hydrostatic pressure, the liquid in the channel breaks through the resistance of the capillary valve and flows downstream of the channel. In this way, the flow and the stop positions of the liquids in these channels may be controlled through the capillary valves 121 and 122.

For instance, the third longitudinal liquid channel 23 is provided with the capillary valve 121 for controlling the flow and the stop position of the liquid, and the capillary valve 121 is higher than the highest liquid level for a solution to be added in the sample chamber 10.

The left side of the test strip chamber 5 communicates with the channel 21, so that when the entire device is turned by a specific angle, the solutions in the sample chamber 10, the reaction chamber 4, and the reagent chamber 8 may enter the test strip chamber 5 through the channel 21. The design of the test strip chamber makes its volume larger, so when the entire device is turned counterclockwise by a specific angle, all the solutions entering may fill a bottom portion of the test strip chamber 5 only. That is, the solutions are in contact with a sample pad portion of a lateral flow test strip in the test strip chamber 5.

The device is provided with channels 61 and 62. When samples and reagents are being added and during subsequent operations, it is necessary to ensure that solutions do not enter channels 61 and 62. After the samples and reagents are added, the sample chamber 10 and the reagent chamber 8 may be isolated from the surrounding environment by using a lid or tape, so that the device becomes a closed system. Further, through the channels 61 and 62, the sample chamber 10, the reagent chamber 8, and the test strip chamber 5 become a connected system, so that it is ensured that the air pressure in each chamber may not interfere with the flow of liquid in the device.

A pneumatic buffer chamber 7 is provided in the device, and the pneumatic buffer chamber 7 communicates with the channels 61 and 62, thereby communicating with the gas inside the device. One side wall of the pneumatic buffer chamber 7 is opened and then closed with a flexible film 13 (as shown in FIG. 3). The flexible film 13 has a large area, so that it can automatically open and expand when it is subjected to pressure without generating stress.

The arrangement of the pneumatic buffer chamber 7 can further improve the anti-contamination capability of the device. In the actual testing of the device, it is often necessary to heat the liquid in the reaction chamber 4 to promote the nucleic acid amplification reaction. For instance, the PCR reaction needs to heat the solution temperature up to approximately 95 degrees. Isothermal amplification, such as LAMP reaction, also requires controlling the reaction solution to be around 65 degrees. Although the heated area may be controlled, such a heating operation may inevitably heat the gas in the reaction device at the same time, thereby causing the gas volume to expand. The design of this device is that during the testing process, the interior of the entire device is under an airtight condition, so that a nucleic acid amplification product is prevented from leaking, thereby avoiding contamination of the amplification product in the environment. However, if the air pressure inside the device is at a positive pressure relative to the environment, it is easy for the gas in the device to leak to the surrounding environment under certain conditions, such as when an operator fails to completely seal the lid after adding a sample to the sample chamber during operation. In order to solve these problems, a pneumatic buffer chamber is arranged in this device. When the gas inside the device is heated and expands, it may generate pressure on the flexible film 13 in the pneumatic buffer chamber and expand the flexible film 13.

In this way, the air pressure balance inside and outside the device is still maintained, and the leakage of gas in the device to the outside that may occur under some extreme conditions is prevented from occurring, and the anti-contamination capability of the entire device is further enhanced.

Further, the arrangement of the pneumatic buffer chamber also ensures that although the interior of the entire device does not communicate with the external environment, the air pressure is balanced with the external air pressure. In this way, air bubbles are prevented from forming in the liquid due to excessively high air pressure inside the device during the heating process, thereby affecting the amplification reaction, and the air bubbles are also prevented from accumulating in the channel to affect the gas flow.

The specific testing implementation process of the disclosure is introduced in the following paragraphs:

### 1. Adding samples and Testing Reagents

A nucleic acid amplification reagent is pre-placed in the reaction chamber 4. According to the requirements of the nucleic acid amplification reagent pre-placed in the

reaction chamber 4, a nucleic acid sample solution that has undergone a standard nucleic acid extraction process is added to the sample chamber 10, or it can also be a nucleic acid sample solution that has undergone simple processing, such as heating or dilution. The amount of nucleic acid sample solution added may be approximately 50 to 1,000 microliters. If the requirements of the subsequent immunoassay test can be satisfied, the amount of nucleic acid sample solution added may be less.

A testing reagent is added to the reagent chamber 8, and the testing reagent is a labeled single-stranded DNA sequence. In the channels 33 and 21 and the test strip chamber 5, the labeled single-stranded DNA sequence in the testing reagent may be combined with an amplified target sequence in a base-complementary form. In this way, the target nucleic acid sequence is marked, so as to facilitate the testing of the immunoassay nucleic acid testing test strip. The amount of testing reagent added may be 10 to 1,000 microliters.

### 2. Nucleic Acid Amplification Reaction

After the nucleic acid sample solution is added to the sample chamber 10, since the reaction chamber 4 is located at the lower left of the sample chamber 10 and communicates with the sample chamber 10 through a channel, under the action of static pressure, the nucleic acid sample solution may enter the reaction chamber 4, and the nucleic acid amplification reaction may obtain a reaction amplification solution.

The reaction chamber 4 itself is communicated with the air above the liquid level in the sample chamber 10 through the channel at the lower left. Further, the capillary channel 11 may also be further provided at an upper left portion of the reaction chamber 4, and the highest end of the capillary channel 11 is higher than the liquid level in the sample chamber 10. In this way, the capillary channel 11 may also provide the function of air pressure communication, so that the nucleic acid sample solution may fill the reaction chamber 4.

Although the sample solution may also enter the capillary channel 11 and channel 23, the volumes of these channels may be basically ignored compared to the reaction chamber 4 in the design. Further, although the sample chamber 10 and the reaction chamber 4 are connected through the channel 24, in this channel 24, the solute components may only move through diffusion. In the time scale of nucleic acid amplification reaction, the mass transfer effect caused by diffusion is small, so that the entire nucleic acid amplification reaction is only carried out in the reaction chamber 4.

The volume of the reaction chamber 4 may be controlled between 3 and 50 microliters in the design. In this way, as long as an appropriate sample is added to the sample chamber 10, according to the design of the device, a corresponding volume of solution may be obtained in the reaction chamber 4 and reacted. This may significantly reduce the consumption of nucleic acid amplification biochemical reagents and lower the costs.

The temperature control of nucleic acid amplification in the reaction chamber 4 may use an external or attached temperature control device. According to needs, PCR reaction may be carried out, and isothermal amplification reaction may also be implemented, common ones include LAMP and RPA.

When the nucleic acid amplification reaction volume is greater than 50 microliters, the reaction chamber 4 may be omitted, and the amplification reaction may be directly performed in the sample chamber 10.

### 3. Nucleic Acid Test Strip Testing of Amplified Products

After the amplification reaction is completed, the device is turned counterclockwise by a specific angle, such as 90 degrees. Herein, under the action of static pressure, the solutions in the reaction chamber 4, the sample chamber 10, and the reagent chamber 8 may pass through the channel 33 and the channel 21 and finally enter the bottom portion of the test strip chamber 5. In the channel and in the bottom portion of the test strip chamber 5, various solutions including the testing reagent, the nucleic acid sample solution, and the reaction amplification solution are mixed.

After being mixed and reaching the bottom portion of the test strip chamber 5, the solutions contact the sample pad portion of the nucleic acid test strip in the test strip chamber 5, so that testing is achieved on the nucleic acid test strip through the immunoassay reaction.

On the one hand, the single-stranded DNA sequence used for labeling in the testing reagent is combined with the target nucleic acid sequence in the reaction amplification solution. On the other hand, due to the mixing of the testing reagent, the nucleic acid sample solution, and the reaction amplification solution, the originally small volume of the reaction amplification solution is diluted and the volume becomes larger, which is conducive to immunoassay testing.

### Implementation Examples of the Disclosure

### 1. Isothermal Amplification and Immunoassay Test Strip Testing of Salmonella

The structure of this example is shown in FIG. 1, and the material used is a polymethyl methacrylate (PMMA) plate with a thickness of 5 mm. The pattern shown in FIG. 1 is processed on one side of the plate using mechanical processing (please note that FIG. 1 is for convenience of illustration and is not drawn based on the actual size. If there is any inconsistency between the graphic representation and the text description, the text description below shall prevail).

The depths of the sample chamber, the reaction chamber, the reagent chamber, and the pneumatic buffer chamber are all 2 mm, and the depth of the test strip chamber is 3 mm. The sample chamber has a width of 4 mm and a height of 40 mm. The distance between an in-chamber dividing plate in the chamber and the top portion of the chamber is 30 mm, and the distance between the right end of a partition plate and the chamber wall is 1 mm. The reaction chamber has a width of 4 mm and a height of 3 mm. The reagent chamber has a width of 4 mm and a height of 50 mm. The distance between the in-chamber dividing plate and the top portion of the chamber is 30 mm, and the distance between the right end of the partition plate and the chamber wall is 1 mm. The test strip chamber has a height of 42 mm and a width of 62 mm. The pneumatic buffer chamber has a width of 15 mm and a height of 10 mm.

The cross-sectional dimensions of the longitudinal channels 21, 22, 23, and 24 are 0.5×0.5 mm, and the length and shape of each channel are set according to actual needs. However, it is necessary to ensure that the top portion of the channel 23 is higher than the highest height of the liquid level of the sample added in the sample chamber. If necessary, the capillary valves may be provided in the channels 22 and 23 to prevent the flow of liquid in the channels. The capillary valve 121 is arranged at a position higher than the highest liquid level for a solution to be added in the sample chamber 10. The capillary valve 122 is arranged at a position higher than the highest liquid level for a solution to be added in the reagent chamber 8.

The cross-sectional dimensions of the transverse channels 31, 32, and 33 are 0.5×0.5 mm, and the shape and length of the channels are determined according to actual needs.

The cross-sectional dimensions of the gas communication channel 61 and 62 are 0.2×0.2 mm, and the shape is determined according to actual needs, so as to ensure the connection between the test strip chamber and the reagent chamber and the connection between the reagent chamber and the sample chamber.

The cross-sectional dimension of the capillary channel 11 is 0.1×0.1 mm, and the shape is determined according to actual needs, so as to achieve the connection between the upper portion of the sample chamber and the reaction chamber.

After the above structure is processed, bonding, adhesion, and other techniques may be used to bond it with another PMMA plate to build a complete chamber. At the corresponding position of the pneumatic buffer chamber 7 of this plate, a rectangular chamber with a width of 15 mm and a height of 10 mm needs to be formed. Next, the surface is sealed with a flexible film with good stretchability and a large area to form a pneumatic buffer chamber.

A cap or adhesive tape may be placed on the top portion of the sample chamber, so that the entire device may be sealed after the sample is added.

This embodiment takes the testing of Salmonella as an example. The invasion protein A (invA) gene of Salmonella is used as the target to perform LAMP amplification reaction and immunoassay test strip testing.

The test strip used in the device is a common nucleic acid testing immunoassay test strip, with streptavidin fixed on the T line and FAM antibodies connected to the colloidal gold.

The added sample is a cultured Salmonella sample with a concentration of 1,000 cfu per ml, which has been extracted with magnetic beads for nucleic acid. The amount of sample added to the sample chamber is 100 microliters.

After the sample is added, the sample chamber may be sealed with tape or a tube cap, etc., to isolate the gas and liquid exchange inside and outside the device and prevent possible aerosol contamination. An external temperature control device or a temperature control device 15 attached to the chip is used to heat the reaction, the temperature is controlled at about 65 degrees. and the reaction is performed for 30 minutes.

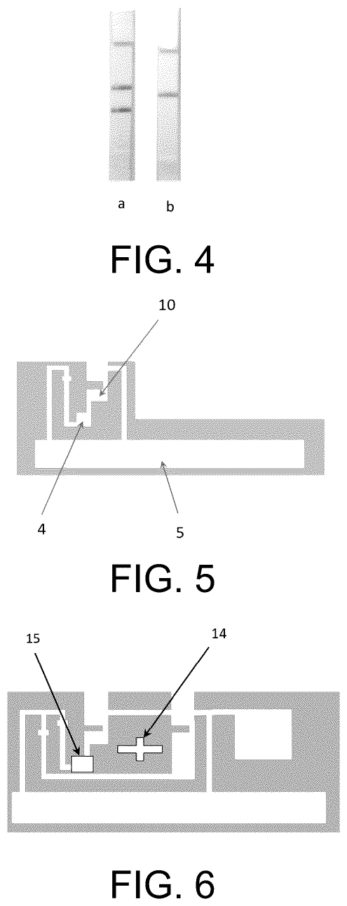

The device is then turned counterclockwise by a specific angle, such as 90 degrees, to achieve flowing and mixing of liquids in the reaction chamber, the reagent chamber, and the sample chamber through the hydrostatic pressure in the device chip. The liquid eventually enters the bottom portion of the test strip chamber and contacts the sample pad of the test strip, and immunoassay begins. Wait 5 to 15 minutes and observe the test strip in the test strip chamber. A positive sample will have two bands, while a negative sample will have only one band (as shown in a and b of FIG. 4, respectively).

### 2. PCR Amplification and immUnoassay Test Strip Testing of SALMONELLA

The device of Example 1 is used, but PCR amplification is performed, so the temperature control device configured may perform temperature cycling between 50 degrees and 95 degrees.

The amplification enzyme used herein is TaKaRa Taq™ HS DNA polymerase, purchased from Bao Biology Engineering (Dalian) Co., Ltd., and the matching reaction buffer is used for lyophilization. F3 and B3 in Example 1 are used as primers for PCR amplification. The 5′ end of F3 is labeled with FAM. The concentration of F3 during PCR amplification is 0.4 μM. while the concentration of B3 is 0.4 μM. This is an asymmetric PCR amplification, and the amplification product naturally generates a single strand, which can be combined with a testing probe to achieve subsequent nucleic acid immunoassay test strip testing. Other PCR amplification conditions are as required by ordinary normal PCR.

The 5′ end of the testing probe is labeled with biotin, and the testing probe is dissolved at a concentration of 0.2 μM, used as a testing reagent, and added to the reagent chamber in an amount of 20 microliters.

After that, turn the device 90 degrees counterclockwise and wait 5 to 15 minutes. Observe the test strip in the test strip chamber. A positive sample will have two bands, while a negative sample will have only one band.

### 3. Isothermal Amplification and Immunoassay Test Strip Testing of SALMONELLA in a Simplified Device

This example uses a simplified device, as shown in FIG. 5. In this device, no reagent chamber is arranged. In the LAMP amplification reaction, the biotin-labeled LB3 reagent is also placed in the lyophilized material in the reaction chamber. Next, the LAMP amplification and test strip testing are carried out as in Example 1.

The advantage of this example is that the reagent chamber in the device is omitted and the reagent adding step is also simplified. However, a possible disadvantage is that if the primers and reaction system are not properly designed, primer dimers and other side reactions between FIB and LB may occur, resulting in a false positive result.

### 4. Performing Testing by Using Automatic Turning Instrument

As shown in FIG. 6, an asymmetric cross-shaped structure is pre-cut in the device chip. Then, during testing, the device chip is placed at the corresponding interface of the steering engine using this structure. LAMP amplification is performed as in Example 1. After the amplification is completed, the automatic turning of the device is achieved by the turning of the steering engine, and the liquid flowing in the device is completed to achieve the immunoassay test strip testing of the amplification result. The turning angle and direction of the steering engine may be precisely controlled according to the needs of the liquid flowing in the device chip.

1. A nucleic acid testing device, comprising
a base plate that is turned during nucleic acid testing, wherein the base plate comprises the following chambers, which are:
a sample reaction chamber for adding and containing a nucleic acid sample solution and a nucleic acid amplification reagent; and
a test strip chamber for lateral flow test strip testing, wherein a nucleic acid test strip is pre-placed in the chamber, the test strip chamber communicates with a sample chamber through a bent channel, and at least one point in the bent channel is higher than a highest liquid level for a solution to be added in the sample chamber.

2. The nucleic acid testing device according to claim 1, wherein
the sample reaction chamber is specifically divided into:
comprising the sample chamber for adding and containing the nucleic acid sample solution, wherein the nucleic acid sample solution is placed in the chamber; and
further comprising a reaction chamber for nucleic acid amplification reaction located below the sample chamber, the nucleic acid amplification reagent for nucleic acid amplification reaction is pre-placed in the chamber, the test strip chamber communicates with the sample chamber through the reaction chamber, an upper end of the reaction chamber communicates with a bottom end of the sample chamber though a liquid channel, and the test strip chamber communicates with the reaction chamber through the bent channel.

3. The nucleic acid testing device according to claim 2, wherein
a capillary valve for controlling flowing and a stop position of a liquid is provided in the bent channel, wherein a position of the capillary valve is set as:
when the device is in a horizontal state, a solution static pressure in the bent channel cannot break through a resistance of the capillary valve, and when the device is in a non-horizontal state, position changes of the sample reaction chamber/sample chamber and the reaction chamber cause the solution static pressure in the bent channel to increase, so that the solution static pressure in the bent channel breaks through the capillary valve and causes the liquid to flow in the bent channel.

4. The nucleic acid testing device according to claim 1, further comprising
a gas communication channel, wherein the test strip chamber communicates with a top portion of the sample reaction chamber/sample chamber after passing through the gas communication channel.

5. The nucleic acid testing device according to claim 1, further comprising
a reagent chamber for accommodating a nucleic acid testing reagent and at a same height as the sample reaction chamber/sample chamber, wherein a testing reagent is added to the chamber, a lower end of the reagent chamber communicates with the bent channel between the test strip chamber and the sample reaction chamber/reaction chamber through the liquid channel, and at least one point in the liquid channel is higher than the highest liquid level for a solution to be added in the sample reaction chamber/sample chamber and a highest liquid level for a solution to be added in the reagent chamber.

6. The nucleic acid testing device according to claim 5, further comprising
further comprising a gas communication channel, wherein the test strip chamber communicates with the sample reaction chamber/sample chamber and a top portion of the reagent chamber through the gas communication channel.

7. The nucleic acid testing device according to claim 5 wherein
each of the sample reaction chamber/sample chamber and the reagent chamber is provided with a partition structure for preventing the solution in the chamber from entering a communication channel at a top portion of the chamber when the chamber is turned.

8. The nucleic acid testing device according to claim 5, further comprising
further comprising a pneumatic buffer chamber for anti-contamination, wherein the pneumatic buffer chamber communicates with the gas communication channel, an opening is provided on one side wall of the pneumatic buffer chamber, and a flexible film is arranged to seal the opening.

9. The nucleic acid testing device according to claim 2, wherein
the reaction chamber is provided with a temperature control device for heating the reaction chamber.

10. A nucleic acid testing method applied to the nucleic acid testing device according to claim 1, comprising
S1: pre-adding the nucleic acid amplification reagent and the nucleic acid sample solution to the sample reaction chamber and directly performing the nucleic acid amplification reaction in the sample reaction chamber; and
S3: obtaining a reaction amplification liquid in the nucleic acid sample solution after the amplification reaction is completed and turning the entire device by an angle in a clockwise direction where a bent channel is arranged, the solutions in the sample reaction chamber and the reagent chamber enter a bottom portion in the test strip chamber after passing through the bent channel under the action of a static pressure and are mixed, so that a labeled single-stranded DNA sequence in the testing reagent is combined with a target nucleic acid sequence in the reaction amplification liquid, and after reaching the bottom portion of the test strip chamber, the mixed solution contacts the nucleic acid test strip in the test strip chamber for immunoassay reaction and testing.

11. A nucleic acid testing method applied to the nucleic acid testing device according to claim 2, comprising
S1: pre-adding the nucleic acid amplification reagent to the reaction chamber, pre-adding the nucleic acid sample solution to the sample chamber, and pre-adding a testing reagent to the reagent chamber;
S2: adding the nucleic acid sample solution to the sample chamber, wherein the nucleic acid sample solution enters the reaction chamber under the action of a static pressure, and the nucleic acid amplification reaction is performed in the reaction chamber; and
S3: obtaining a reaction amplification liquid in the reaction chamber after the amplification reaction is completed and turning the entire device by an angle in a clockwise direction where a bent channel is arranged, wherein the solutions in the reaction chamber, the sample chamber, and the reagent chamber enter a bottom portion in the test strip chamber after passing through the bent channel under the action of the static pressure and are mixed, so that a labeled single-stranded DNA sequence in the testing reagent is combined with a target nucleic acid sequence in the reaction amplification liquid, and after reaching the bottom portion of the test strip chamber, the mixed solution contacts the nucleic acid test strip in the test strip chamber for immunoassay reaction and testing.

12. The nucleic acid testing device according to claim 2, further comprising a gas communication channel, wherein the test strip chamber communicates with a top portion of the sample reaction chamber/sample chamber after passing through the gas communication channel.

13. The nucleic acid testing device according to claim 2, further comprising
a reagent chamber for accommodating a nucleic acid testing reagent and at a same height as the sample reaction chamber/sample chamber, wherein a testing reagent is added to the chamber, a lower end of the reagent chamber communicates with the bent channel between the test strip chamber and the sample reaction chamber/reaction chamber after passing through the liquid channel, and at least one point in the liquid channel is higher than the highest liquid level for a solution to be added in the sample reaction chamber/sample chamber and a highest liquid level for a solution to be added in the reagent chamber.

14. The nucleic acid testing device according to claim 13, further comprising
further comprising a gas communication channel, wherein the test strip chamber communicates with the sample reaction chamber/sample chamber and a top portion of the reagent chamber after passing through the gas communication channel.
